(19) United States
(12) Patent Application Publication
(10) Pub. No.: US 2005/0256204 A1
(43) Pub. Date: Nov. 17, 2005

(54) Topical phenyl-epinephrine Rosacea treatment

(76) Inventor: Patrick H. Bitter SR., Los Gatos, CA (US)

Correspondence Address: LAW OFFICES OF THOMAS E. SCHATZEL;A Professional Corporation, Suite 240, 16400 Lark Avenue, Los Gatos, CA, 95032-2547, US

(21) Appl. No.: 10/843,773

(22) Filed: May 11, 2004

(51) Int. Cl. A61K 31/137; A61N 1/30

(52) U.S. Cl. 514/649; 604/20

(57) ABSTRACT

A near-permanent skin treatment includes photothermolysis of reddened facial skin to induce ischemia. Reperfusion of the photothermolysis treated skin is inhibited by following with regular applications of phenyl-epinephrine carried in a lotion until vascular necrosis is complete. Alternatively, a temporary treatment for reddened facial skin includes only cosmetic as-needed applications of phenyl-epinephrine carried in lotion to induce vasoconstriction in Rosacea and other similarly embarrassing skin disorders.

### BACKGROUND OF THE INVENTION

1. Field of the Invention

The present invention relates to medical treatment of Rosacea and related skin disorders. In particular, it relates to using topically applied vasoconstrictors for user-selective ischemia effects for temporary relief of embarrassing outbreaks.

2. Description of the Prior Art

As many as seventy million Americans have an undesirable redness and/or sun damage of the face. The more common causes are Rosacea, sun exposure, alcohol, medications, stress, emotional causes, time and aging of the skin, repeated blushing or flushing of the skin. Such conditions are seen most commonly in fair skinned individuals of northern European descent. Some sufferers also experience Erythrophobia, e.g., a morbid fear of having a red face and being embarrassed in public by it.

Rosacea is a common skin disease. It is hereditary, chronic skin disorder that causes slight to severe redness in the face that may come and go on its own. If left untreated, Rosacea skin can become inflamed and erupted. The affected skin tissue can swell and thicken, and be very sensitive to the touch, common cosmetics, and medical lotions. Ocular Rosacea includes those Rosacea sufferers with symptoms in their eyes.

Sufferers of Rosacea often lead lives that are controlled by their reluctance to appear in public. For some, this can lead to quitting jobs, staying home, hiding from the world, and feeling handicapped. A large medical and pharmaceutical industry has developed to market products and services to Rosacea patients.

Rosacea is visible when the arterioles, capillaries, and venules in facial skin dilate. These tiny microvessels are close to the surface of the skin, so the blood in them becomes visible through the thin semi-transparent covering. The skin breaks out in papules, e.g., blotchy red areas. In non-severe outbreaks, such papules are red solid elevated inflammatory skin lesions without pus.

Treatments for dilated blood vessels, especially varicose veins, include sclerotherapy and surgical therapy. In sclerotherapy, a sclerosing solution, such as hypertonic saline or surfactants, is injected into the involved blood vessels, and results in deformation of the vascular structure. Surgical therapy involves extensive ligation and stripping of the greater and lesser saphenous veins. However, administration of these therapies usually requires high technical skill. Furthermore, the common patients, fear of needles and surgical procedures prevents many from seeking these treatments.

Laser, and intense non-coherent light sources, have been used in photothermolysis therapy to treat dilated blood vessels, such as Rosacea. Selectively-absorbed colored light is used to damage the vessels while sparing the surrounding tissues. However, reperfusion of treated blood vessels reduces the effectiveness of the treatment. Reperfusion is the restoration of blood supply to tissue which is ischemic due to decrease in normal blood supply.

In the prior art, multiple laser treatments are often required due to reperfusion of the treated vessels. Such reperfusion is also undesirable as it can lead to clotting and thrombolysis. Photothermolysis therapy is in common use, despite the relatively high cost, number of treatments needed, and the risk of post-treatment pigmentation.

For example, U.S. Pat. No. 6,306,130, issued Oct. 23, 2001, to Anderson, et al., describes methods for eliminating blood vessels in skin by non-invasively heating the walls of a blood vessel to 60° C., and then forcibly pressing together or collapsing the walls for a time sufficient to allow them to cool. The vessel walls become adhered or “welded” together, resulting in closure of the vessel lumen, irreversible necrosis and degradation, and thus removal of the blood vessel during the subsequent healing. The new non-invasive methods greatly reduce reperfusion of a treated vessel, and substantially improve the efficacy of conventional photothermolysis therapy.

Such is a non-invasive method of inducing necrosis and degradation of a blood vessel, e.g., a dilated vein, in a tissue, such as skin, by non-invasively heating walls of the blood vessel to a temperature of at least about 60° C., and collapsing the blood vessel by applying pressure to allow welding together of the blood vessel walls. The blood vessel thereafter proceeds into necrosis.

The energy source can be an optical source, such as a pulsed or scanned optical source, a laser, or a xenon flash lamp. Ultrasound and radio frequency energy sources can also be used. For example, the optical source can be a visible or near-infrared optical source that emits in a wavelength range of 500-1100 nanometers and delivers optical energy with exposure durations of, e.g., 1 to 100 or 5-50 milliseconds.

The pressure has to be applied at the right time, e.g., after heating is completed, for example, when blood within the vessel is vaporized. For example, pressure can be applied within one thermal relaxation time of the blood vessel after the vessel walls are heated to at least 60° C. The pressure must also be maintained for an adequate time, e.g., at least one thermal relaxation time of the blood vessel. Pressure can be applied by mechanical compression, hydraulic compression, or pneumatic compression.

The present inventor, Patrick Bitter Sr., M.D., developed a cosmetic treatment marketed under his trademark, PHOTOFACIAL. Such treatment includes exposing the facial skin of a patient to a multichromatic intense pulsed light (IPL). The IPL emits an intense pulse of light that includes many wavelengths and respective intensities. Selective photothermolysis matches particular wavelengths included in the light to the thermal relaxation time of the target, and the absorptive wavelength of the target chromophore. The different tissues in the skin have different colors, opacities, and therefore different absorption properties that can be used to select target tissues like red blood vessels. The specific profile of wavelengths and intensities is adjusted to balance hemoglobin and melanin absorption. The PHOTOFACIAL treatment has typically been repeated at intervals of approximately three weeks for a series of five treatments.

Alternative topical treatments have included nitric oxide inhibitors.

The prior art includes the use of phenyl-epinephrine as a vasoconstrictor for the vessels in the cornea of horses as a preparation for eye surgery. Brian Gilger, DVM, MS, Dipl. ACVO, founder of the Equine Ophthalmologic Service at North Carolina State University, made a presentation at the 2002 American Association of Equine Practitioners Convention titled, “How to Prepare for Ocular Surgery in the Standing Horse.” He reports that a topical anesthetic (proparacaine HCl) and phenyl-epinephrine were applied to enhance ocular surface anesthesia and to constrict blood vessels in the cornea and conjunctiva to enhance hemostasis, e.g., stopping of bleeding, and visibility during surgery.

### SUMMARY OF THE INVENTION

Briefly, a near-permanent skin treatment embodiment of the present invention includes photothermolysis of reddened facial skin to induce ischemia. Reperfusion of the photothermolysis treated skin is inhibited by following with regular applications of phenyl-epinephrine carried in a lotion until vascular necrosis is complete. Alternatively, a temporary treatment for reddened facial skin includes only cosmetic as-needed applications of phenyl-epinephrine carried in lotion to induce vasoconstriction in Rosacea and other similarly embarrassing skin disorders.

An advantage of the present invention is that a treatment is provided for Rosacea suffers that is safe and effective.

Another advantage of the present invention is that a skin lotion is provided that can provide effective temporary relief and can be sold over-the-counter for use by patients on an as-needed basis.

A further advantage of the present invention is that treatment is provided in which the number of photothermolysis sessions needed is reduced.

These and other objects and advantages of the present invention will no doubt become obvious to those of ordinary skill in the art after having read the following detailed description of the preferred embodiments which are illustrated in the various drawing figures.

### IN THE DRAWINGS

FIG. 1 is a flowchart diagram of a skin treatment embodiment of the present invention; and

FIG. 2 is a graph diagram of the wavelength versus absorption properties of respective cutaneous chromophores subjected to the treatment of FIG. 1.

### DETAILED DESCRIPTION OF THE PREFERRED EMBODIMENT

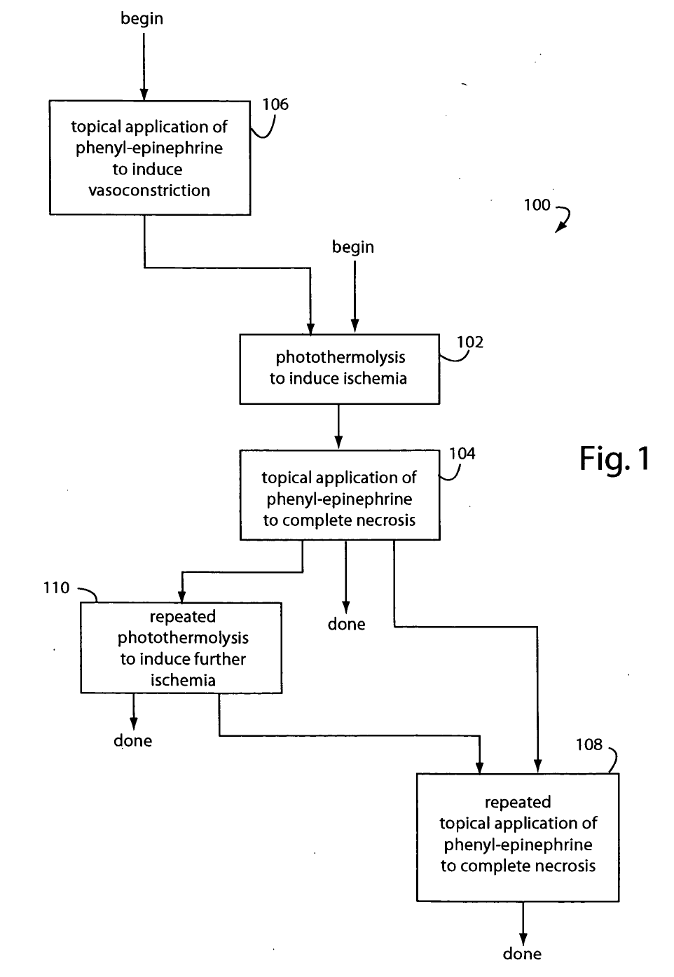

FIG. 1 represents a reddened skin treatment embodiment of the present invention, and is referred to herein by the general reference numeral 100. Such treatment 100 begins with a selective photothermolysis of cutaneous chromophores, in a step 102. The cutaneous chromophores in facial skin include hemoglobin, oxyhemoglobin, and melanin.

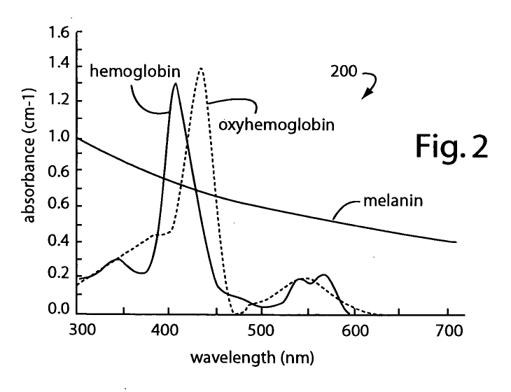

The relative absorptions of light energy by these respective cutaneous chromophores are represented in FIG. 2. (Information source: Lebedev Physics Institute, published at http://yachroma.com/slcEng.htm) Using selective photothermolysis to remove abnormal blood vessel requires the light wavelengths used to coincide with the yellow absorption pick of hemoglobin and oxyhemoglobin, e.g., 576 nm. The light spot must be sized for the diameter of the abnormal vessels, about 0.5 mm. The light pulse duration must be less than thermal relaxation time of vessels. And, the light power must be just sufficient enough for adequate photothermolysis, but not any more.

The photothermolysis is induced, e.g., with a copper laser, or a xenon flash lamp source of intense multichromatic pulsed light. The wavelengths of light chosen for use are in particular absorbed by the red blood ingrained by vascular tissue cells. The resultant heating produces ischemia (blood-supply distress) in these vascular tissue cells. In a step 104, a topical application of phenyl-epinephrine provides for a final necrosis of the ischemic vascular tissue cells.

A preliminary step 106 may be included in treatment 100. A preparatory topical application of phenyl-epinephrine provides for pre-photothermolysis vasoconstriction of the vascular tissue cells. Such reduces the cross-sectional areas of the ischemic vascular tissue cells and makes it easier to close up the vessel walls because they begin photothermolysis already partially closed.

The photothermolysis step 102 may be implemented by exposing the skin to intense pulsed light (IPL) from a non-coherent xenon flash light source, or exposing the skin to laser light. For example, a copper laser source.

At a minimum, treatment 100 comprises only steps 102 and 104. In alternative embodiments of the present invention, a subsequent step 108 repeats the topical application of phenyl-epinephrine until a final necrosis of the ischemic vascular tissue cells occurs. In further alternative embodiments, a secondary photothermolysis 110 is used to inhibit reperfusion of the ischemic vascular tissue cells. Such subsequent step of secondary photothermolysis of the skin can be included to increase the population of the ischemic vascular tissue cells.

The topical applications of phenyl-epinephrine used in steps 104, 106, and 108, can include the use of a skin lotion comprising phenyl-epinephrine in solution with a strength sufficient for transdermal absorption by non-mucous membrane skin tissues. Such phenyl-epinephrine is the only active ingredient, and it is preferably compounded with a water-based gel. Such gel provides for holding the phenyl-epinephrine in contact with a user's skin for a minimal residence time sufficient to induce vasoconstriction. In tests that were conducted that produced the desired vasoconstriction, the phenyl-epinephrine had a strength of at least one percent.

It was discovered that topical applications of phenyl-epinephrine in a cosmetic lotion would provide temporary relief of skin reddening symptoms of Rosacea and other facial skin disorders. For example, using step 106 alone. Further embodiments of the present invention therefore comprise phenyl-epinephrine cosmetic skin lotion. The skin lotion is applied as needed by the user, e.g., just before going out in public.

It is expected that the inclusion of a transdermal carrier with such skin lotion would allow lower strengths of phenyl-epinephrine to be used effectively. For example, less than 1% active ingredient. Conventional transdermal carriers known to artisans include polymethacrylic acid (PMA), carbopol, polyethylene glycol 8000 (PEG), propylene glycol (PG), water, alcohol, acetone, caprylic acid, caproic acid, oleic acid, lauric acid, isopropyl myristate, triethanolamine, or mixtures thereof. However, some of these may not be suitable for use on patients that have very sensitive skin or allergies. Tests would be needed to select suitable transdermal carriers for specific patients and users.

Although the present invention has been described in terms of the presently preferred embodiments, it is to be understood that the disclosure is not to be interpreted as limiting. Various alterations and modifications will no doubt become apparent to those skilled in the art after having read the above disclosure. Accordingly, it is intended that the appended claims be interpreted as covering all alterations and modifications as fall within the “true” spirit and scope of the invention.

1. A reddened skin treatment, comprising:
a photothermolysis of skin that produces ischemia in vascular tissue cells; and
a topical application of phenyl-epinephrine providing for a final necrosis of said ischemic vascular tissue cells.

2. The treatment of claim 1, further comprising the preliminary step of:
a preparatory topical application of phenyl-epinephrine providing for pre-photothermolysis vasoconstriction of said vascular tissue cells for reducing the cross-sectional areas of said ischemic vascular tissue cells.

3. The treatment of claim 1, wherein:
the step of photothermolysis of skin includes exposing said skin to intense pulsed light (IPL) from a non-coherent flash light source.

4. The treatment of claim 1, wherein:
the step of photothermolysis of skin includes exposing said skin to laser light.

5. The treatment of claim 1, further comprising the subsequent step of:
a secondary photothermolysis of said skin to inhibit reperfusion of said ischemic vascular tissue cells.

6. The treatment of claim 1, further comprising the subsequent step of:
a secondary photothermolysis of said skin to increase the population of said ischemic vascular tissue cells.

7. The treatment of claim 1, further comprising the subsequent step of:
repeating the topical application of phenyl-epinephrine until a final necrosis of said ischemic vascular tissue cells occurs.

8. A cosmetic skin lotion, comprising:
phenyl-epinephrine in solution with a strength sufficient for transdermal absorption by non-mucous membrane skin tissues;
a water-based gel providing for holding the phenyl-epinephrine in contact with a user's skin for a minimal residence time sufficient to induce vasoconstriction;
wherein, temporary relief is provided to reduce skin reddening.

9. The lotion of claim 8, further comprising:
a transdermal carrier providing for delivery of molecules of the phenyl-epinephrine to vascular tissue cells in said user's skin.

10. The lotion of claim 8, wherein:
the phenyl-epinephrine is the only active ingredient and has a strength of at least one percent.
